(19) United States
(12) Patent Application Publication
(10) Pub. No.: US 2025/0057700 A1
(43) Pub. Date: Feb. 20, 2025

(54) Absorbent Article with Release Adhesive

(71) Applicant: Edgewell Personal Care Brands, LLC, Chesterfield, MO (US)

(72) Inventor: Joseph Garthaffner, Doswell, VA (US)

(21) Appl. No.: 18/722,760

(22) PCT Filed: Dec. 21, 2022
(86) PCT No.: PCT/US22/82120

§ 371 Date: Jun. 21, 2024

Related U.S. Application Data
(60) Provisional application No. 63/292,760, filed on Dec. 22, 2021.

(51) Int. Cl. A61F 13/15; A61F 13/514

(52) U.S. Cl. CPC A61F 13/15203; A61F 13/514

(57) ABSTRACT

An absorbent article for menstrual and/or incontinence purposes, having evenly applied pressure-sensitive adhesive on the backside of the absorbent article. The absorbent article optionally has embossing creating a varied topography along the body facing side of the absorbent article, including the cover layer and optionally the inner transfer layer and/or the core layer(s). The absorbent article includes a release material or an absorbent article having releasable adhesive applied thereto, where thereafter, the absorbent article is applied to the pressure-sensitive adhesive on the release material by way of a nip including a lobed roller. An apparatus and method are provided herein supporting the manufacture of such absorbent articles.

### CROSS-REFERENCE TO RELATED APPLICATIONS

This application claims priority to U.S. Provisional Patent Application Ser. No. 63/292,760, filed Dec. 22, 2021, the entirety of which is incorporated by reference herein.

### BACKGROUND

The present disclosure relates to an absorbent article for menstrual and/or incontinence purposes. Specifically, the present disclosure relates to absorbent articles having a backsheet (or backside) with adhesive enabling the absorbent article to stick to a release material such as a wrapper, overwrap or release paper, be removed therefrom and then applied to an undergarment, and thereafter be removed therefrom.

Current absorbent products have a number of ways they can be improved. Some current absorbent products are manufactured on equipment that requires release paper to be applied as the manufacturing equipment is not designed to handle materials with different material properties. Such equipment leads to products ultimately having both release paper and a wrapper thereby adding cost and increasing the number of items that might ultimately be discarded by the consumer. Some current absorbent products are equipped with only a wrapper, where the wrapper acts as a protective barrier and the release paper to which the adhesive on the backside of the absorbent product is temporarily adhered to during storage and prior to use. Current manufacturing equipment is able to accommodate the latter product configuration where the product is substantially flat, or said differently, is without embossing, as equipment cannot evenly apply pressure sensitive adhesive on a product having topographical variation [on the cover or body-facing side of the product and/or potentially within at least portions of the interlayers or core of the product]. Such products lack features such as protrusions and channels that can increase comfort for the user while wearing the product, can improve mechanical properties, and can improve absorbent characteristics of the product. Such products rely on visual ques to communicate intended benefits to the consumer given the inability to have embossing. In summary of and in addition to the aforementioned, current absorbent products can be difficult to manufacture and/or costly, as they require multiple components often made by different manufacturers. There is a need to reduce complexity and costs while maximizing comfort and/or performance features of these products, and hopefully improving the environmental impact of these products.

### SUMMARY

An absorbent article for menstrual or incontinence purposes is provided, such as a napkin, including a pad, liner, maxi or ultra-thin product. The absorbent article has a cover layer, and absorbent core structure, and a backsheet that is generally hydrophobic thereby preventing moisture from travelling through the absorbent article and onto the underlying garment (underwear, pants, etc. . . . ) The absorbent article optionally includes a transfer layer. In other embodiments, the properties of the transfer layer have been designed into the cover layer and/or the absorbent core. In such latter embodiments, the overall thickness of the pad is optionally reduced. In such latter embodiments, the overall thickness of at least one of the cover layer and the absorbent core is increased such that the thickness of the layer is greater than prior art products but still achieves a reduced thickness product. While such embodiments as contemplated throughout the present disclosure could be used in numerous forms to achieve a reduction in thickness, an ultra-thin is preferred.

The absorbent article has a length, width and thickness, where the length is between about 150 mm to about 330 mm, and the width varies between about 45 mm to about 150 mm (at its widest portion). The total thickness of the cover, the optional transfer layer, and the absorbent core, is between about 0.9 mm and about 5 mm, or between about 1.5 mm and about 3.3 mm, and more preferably between 2.5 mm and about 3.3 mm. The cover, the optional transfer layer, and absorbent core, has a total weight of between about 1.3 to about 23 g, or between about 1.3 g and 6 g, or between about 1.3 g and about 2.8 g, or between about 7 g and about 23 g, or between about 7 g and about 10 g. The cover, the optional transfer layer, and absorbent core, has a total basis weight (gsm) of less than about 240 gsm, or between about 170 gsm and about 220 gsm, or between about 180 gsm and about 200 gsm.

Optionally, the cover layer is embossed having one or more protrusions separated by one or more channels. The cover layer has a first body facing height and a second body facing height that is different from the first body facing height. The cover layer optionally includes a honeycomb embossed cover, the honeycomb being a repeating pattern of protrusions and channels across at least a portion of the absorbent article, such as a first end region, a second end region, and/or a central region, and/or a first lateral region, a central length region, and/or a second lateral region that is opposite the first lateral region.

The absorbent core has a total weight of between about 2 g and about 5 g, or between about 3 g and about 4 g, or about 3.3 g to about 3.5 g. Having a more robust core material is preferred where the cover material is reduced in weight, where such combination of the core and cover enable a reduced thickness absorbent article. For example, the total thickness of the absorbent article is less than about 3 mm. Such thickness, in combination with structure and materials, enables and imparts advantageous properties and features contemplated throughout the present disclosure.

Super absorbent material comprises between about 10% and about 50% of the entire absorbent core, or between about 15% and about 50% of the entire absorbent core, or between about 15% and about 25% of the entire absorbent core. Pulp or cellulosic fiber content is between about 50% and about 90% of the entire absorbent core, or between about 50% and about 85%.

The absorbent article includes positioning adhesive applied to the backsheet and/or the release material (such as a wrapper, overwrap, or release paper). The terms “positioning adhesive”, “pressure-sensitive adhesive” and “releasable adhesive” are interchangeable for the purposes of the present disclosure. The positioning adhesive includes a pattern such as but not limited to: multiple stripes (or lines) that are covered by one or more pieces of release paper until the absorbent article is ready to be applied to a garment for use. In some embodiments, between 1 and between and 50 lines of adhesive are applied, such that the stripes run parallel to the length of the absorbent article or are perpendicular to the length of the absorbent article. In certain embodiments, between 12 and 50 stripes of adhesive are applied perpendicular to the length of the absorbent article.

In some embodiments, between 1 and between about 50 lines of adhesive are applied, such that the stripes run the parallel to the length of the absorbent article or are perpendicular to the length. In some embodiments, between 2 and 5 lines of adhesive are applied. In some embodiments, 2 lines of adhesive are applied. In alternate embodiments, positioning adhesive is applied in a rectangular or square pattern, at least about the ends of the absorbent article. In certain embodiments, between 20 and 28 stripes are applied perpendicularly to the length of the absorbent article or angled with respect to the length of the absorbent article. In certain embodiments, between 30 and 38 stripes are applied perpendicularly to the length of the absorbent article. In certain embodiments, between 40 and 48 stripes are applied perpendicularly to the length of the absorbent article. Stripes or lines can include multiple discrete segments. In such embodiments, the at least two adhesive stripes have a stripe width between 4 mm and about 40 mm, or between about 4 mm and about 18 mm. In such embodiments, the at least two adhesive stripes have a stripe length of between about 180 mm to about 290 mm, or between about 180 mm and about 200 mm, or about 190 mm, or between about 220 mm and about 250 mm, or about 240 mm, or between about 250 mm and about 290 mm, or about 270 mm.

In alternate embodiments, positioning adhesive is applied in a rectangular or square pattern, at least about the ends of the absorbent article. In some embodiments, stripes of adhesive are applied where at least one of the stripes has a length that is different from at least one other stripe of adhesive. In some embodiments, a plurality of stripes that are oriented perpendicular to the length of the absorbent article, where a first stripe situated most proximal of the stripes to the upper edge of the absorbent article is shorter than the stripes situated inward of the first stripe. In some embodiments, a plurality of stripes that are oriented perpendicular or angled to the length of the absorbent article, where a last stripe situated most proximal of the stripes to the lower edge of the absorbent article is shorter than the stripes situated inward of the last stripe.

The adhesive stripes are spaced away from each other by portions of the back layer of the absorbent article. Such areas where there is no adhesive are referred to as adhesive free zones. Adhesive free zones can have a length less than (where the line is arcuate or includes the intersection of more than one line), equal to or greater than the length of the adhesive stripe. Adhesive free zones can have a width of between about 1 mm and about 10 mm, or between about 2 mm and about 7 mm, or between about 2 mm and about 6 mm, or between about 4 mm and about 6 mm.

In some embodiments, the adhesive is applied to the back layer of the absorbent article in an amount between about 20% and about 50% of the total surface area of the back layer of the absorbent article.

In some embodiments, the adhesive is applied to the article facing side of the wrapper or the release material in an amount between about 20% and about 50% of the total surface area of the article facing side of the wrapper.

In some embodiments, adhesive is applied as taught by the present disclosure in stripes onto the release material (e.g, the wrapper, overwrap, or the release paper) of the absorbent article. The wrapper is then applied to the back layer of the absorbent article.

In some embodiments, the cover layer and absorbent core are assembled using laminating adhesive. In other embodiments, laminating adhesive is not required for such assembly, rather the products are assembled via heat and/or mechanical crimping.

The absorbent article optionally has a simplified manufacturing process. In some embodiments, the absorbent article does not require a release paper. As such, only one release material (i.e., just the wrapper or overwrap only, and not also a release paper) needs to be applied and is discarded after the user applies the product to the user's undergarment. Such simplified structure is advantageous from a cost perspective.

Embodiments of the absorbent article as taught by the present disclosure are advantages over the prior art. For example, some embodiments of the absorbent article have embossing, which is difficult to achieve with a single release material, the wrapper or overwrap, and not also a release paper).

The absorbent article according to the present disclosure has an adhesive strength of at least 100 gf, or between about 100 gf and about 300 gf, or between about 100 gf and about 250 gf.

A method of applying adhesive to wrapper and onto a backside of an absorbent article is provided. The method provides a wrapper material and an absorbent article. The absorbent article is formed from at least a cover layer, a core, and a backsheet together, and are assembled together either by pressure, heat, adhesive, or a combination thereof. The cover layer may be formed to the backsheet, and/or each layer may be attached directly to its immediately adjacent layer. The absorbent article has embossing such that it has one or more protrusions separated by one or more channels.

The release material (e.g. the wrapper, overwrap, or release paper) has adhesive applied in a pattern according to the present disclosure. The adhesive is applied by one or more nozzles. The nozzles are configured to spray one or more lines or patterns of adhesive onto the wrapper material or release paper material according to the present disclosure. After the adhesive applying step, the wrapper material or release paper material is conveyed by at least a transfer drum into a nip having a gap size of between about 0.5 mm and about 4 mm, or between about 0.5 mm and about 3 mm, or about 1 mm. The wrapper material or release paper material is conveyed such that the width of the material corresponds to the length of the at least one absorbent article. The gap size can be adjusted to suit the size (i.e., the thickness) of the absorbent article. The gap size can be adjusted to accommodate different sized rollers suitable for different sized absorbent articles.

Simultaneously, at least one absorbent article is conveyed into a nip, where the at least one absorbent article is flat (or is not yet folded). In some embodiments, the length of the at least one absorbent article is aligned with the width the belt or rotating drum. In certain embodiments, the at least one absorbent article is transferred by a belt or by vacuum. In one embodiment, a rotating transfer drum applies a vacuum pressure to at least one absorbent article.

As the at least one absorbent article is applied to the adhesive on the release material within the nip, pressure is applied by a lobed roller thereby providing a secured but releasable connection between the release material and the backside of the at least one absorbent article. The at least one absorbent product and the release material engage and pass through the nip created by the lobed roller and the transfer roller, where the transfer roller is generally opposite the lobed roller such that each of the two rollers is on the opposite side of the at least one absorbent product and the wrapper material. The at least one absorbent product and the release material engage the lobed roller at the first portion of the lobed portion. The lobed portion of the roller is sized to be generally the width of the absorbent article. The first portion of the lobed roller has a lead-in to mitigate against the at least one absorbent article from bunching and causing it to not properly sit on or mate with the adhesive applied to the release material. The lobed roller has a pitch of between about 120 mm to about 150 mm, or about 130 mm to about 140 mm, or about 131 mm to about 136 mm.

The lobed roller rotates at a speed substantially equal to the conveyed speed of the wrapper material or release paper material. In embodiments where the at least one absorbent article is conveyed on a belt, the lobed roller rotates at a speed substantially equal to the conveyed speed of the at least one absorbent article. In embodiments where the at least one absorbent article is conveyed by a transfer drum (using a vacuum), the rotational speed of the lobed roller equals the rotational speed of the transfer drum.

The lobed roller is structured to have a first circumference and a second circumference, where the first circumference has a maximum diameter of the lobed portion of the roller, where the second circumference has a minimum diameter of the trough. The first circumference forms a circumscribed circle about the entire roller including at least one lobed portion and at least one trough. The second circumference forms an inscribed circle about the entire roller. In some embodiments, there is at least two lobed portions and at least two troughs, where the troughs separate each of the lobed portions.

An apparatus for applying a wrapper material or release paper material to an absorbent article is provided. The apparatus includes a drum having a rolling surface. The rolling surface is non-circular where a portion of rolling surface has a maximum diameter that defines a circumscribed circle. The rolling surface is optionally lobed such that a portion of the rolling surface has a diameter less than the maximum diameter of the rolling surface defined by the lobed portion of the rolling surface. The rolling surface is optionally cammed such that a portion of the rolling surface has a diameter less than the maximum diameter of the rolling surface defined by the cammed portion of the rolling surface.

The process and apparatus of the present disclosure provide improved consistency of adhesive within the adhesive stripe(s) for embossed absorbent articles. Prior processes and apparatus' were unable to consistently apply adhesive in a configuration of one or more stripes, to which the present disclosure is able to achieve. As such, the absorbent article of the present disclosure includes one or more adhesive stripes that has a substantially uniform amount of adhesive applied throughout an individual adhesive stripe, in at least one of the length and thickness of the adhesive stripe. As such, the absorbent article of the present disclosure includes one or more adhesive stripes that has a substantially uniform amount of adhesive applied throughout all of the one more adhesive stripes on the absorbent article, in at least one of the length and thickness of such one or more adhesive stripes.

Other exemplary embodiments of the present disclosure are provided below:

An absorbent article having a length, a width, and a thickness, the length and width being in the x-y plane, comprising:
a cover layer having a body facing surface and a lower cover surface opposite to the body facing surface;
an absorbent core adjacent to the lower cover surface; and
a backsheet layer having a backsheet upper surface and a garment-facing surface opposite the backsheet upper surface, the garment-facing surface having at least some portions covered by a releasable adhesive;
wherein at least one of the cover layer and the absorbent core are embossed thereby creating a first body facing height that is greater than a second body facing height;
wherein the garment-facing surface has a releasable adhesive is applied in a plurality of stripes; and
wherein the plurality of stripes is between two stripes and fifty stripes;
further comprising a release material comprising a semi-siliconized or a fully siliconized film material;
wherein absorbent article is embossed to have at least one channel and up to about 25 channels;
wherein cover layer is embossed such that there are embossed protrusions on the body facing surface with respect to a cover layer base height;
wherein the embossed protrusions have a protrusion height of between about 0.020 inches (0.5 mm) and about 0.040 inches (1.0 mm), and a protrusion width of between about 0.020 inches (0.5 mm) and about 0.040 inches (1.0 mm); and
further comprising at least one channel separating the embossed protrusions, wherein the channels have a channel width of between about 0.0125 inches (0.31 mm) and about 0.0375 inches (0.95 mm).

An absorbent article having a length, a width, and a thickness, the length and width being in the x-y plane, comprising:
a cover layer having a body facing surface and a lower cover surface opposite to the body facing surface;
an absorbent core adjacent to the lower cover surface; and
a backsheet layer having a backsheet upper surface and a garment-facing surface opposite the backsheet upper surface, the garment-facing surface having at least some portions covered by a releasable adhesive;
a release material being generally rectangular and having an upper edge, a left-most edge, a right-most edge generally opposite the left-most edge, and a lower edge generally opposite the upper edge, the release material having an article facing side and an outer side opposite to the article facing side wherein the article facing side and the outer side are defined by the upper edge, lateral edges, and lower edge, the release material having a release material length that is equal to or greater than the length of the absorbent article, the release material having a release material width that is equal to or greater than the width of the absorbent article, the release material comprising a semi-siliconized or fully-siliconized material or coating;
wherein at least one of the cover layer and the absorbent core are embossed thereby creating a first body facing height that is greater than a second body facing height;
wherein the release material has a releasable adhesive is applied in a plurality of stripes,
wherein the plurality of stripes is at least two stripes and up to about 50 stripes,
wherein the plurality of stripes have an adhesive surface area of up between about 20% and up to about 50% of the total surface area of the article facing side of the release material;
wherein the plurality of stripes have an adhesive surface area of between about 20% and up to about 50% of the total surface area of the garment-facing surface.

A method for applying adhesive to at least one ultra-thin absorbent article, comprising the steps of:
assemble the at least one ultrathin absorbent article from at least a cover layer, a core having one or more portions or layers, and backsheet, wherein the backsheet has a garment facing surface that provides a backside of the absorbent article;
wherein the cover layer is applied to and is adjacent the core, and the core is applied to and adjacent the backsheet, wherein the cover layer, core and backsheet are assembled into the at least one ultra-thin absorbent article by at least one of pressure, heat, adhesive, or a combination thereof;
wherein the at least one ultra-thin absorbent article is assembled such that the cover layer (11) has a varied topography with one or more protrusions and one or more channels;
a release material is provided and is conveyed past one or more nozzles, wherein the nozzles spray adhesive onto the release material in one or more lines or patterns as the release material is conveyed;
convey the release material by at least a transfer drum into a nip having a gap size of between about 0.5 mm and about 5 mm;
wherein the release material is conveyed such that the width of the release material corresponds to the length of the at least one ultra-thin absorbent article;
simultaneously, convey the at least one ultra-thin absorbent article is conveyed into a nip on a transfer belt, where the at least one ultra-thin absorbent article is generally and substantially flat or not in a folded condition;
wherein the length of the at least one ultra-thin absorbent article is aligned with the width the transfer belt or rotating drum; and
as the at least one ultra-thin absorbent article is applied to the adhesive on the release material within the nip, pressure is applied by a lobed roller thereby providing a secured but releasable connection between the release material and the backside of the at least one ultra-thin absorbent article;
wherein the at least one ultra-thin absorbent article and the release material engage and pass through the nip created by the lobed roller and the transfer drum;
wherein the transfer drum is generally opposite the lobed roller such that each of the transfer drum and lobed roller are on the opposite side of the at least one ultra-thin absorbent article and the release material;
wherein the at least one ultra-thin absorbent article and the release material engage the lobed roller at the first portion of the lobed portion;
wherein the lobed portion of the lobed roller is sized to be generally the width of the at least one ultra-thin absorbent article;
wherein the first portion of the lobed roller has a lead-in to mitigate against the at least one ultra-thin absorbent article from bunching and causing the at least one ultra-thin absorbent article to not properly sit on or mate with the adhesive applied to the release material; and
wherein the lobed roller rotates at a speed substantially equal to the conveyed speed of the release material.

### BRIEF DESCRIPTION OF THE DRAWINGS

FIG. 1 shows an angled top view of an embodiment of the present disclosure;

FIG. 1a shows a detail schematic view of an embodiment of a cover layer of the present disclosure;

FIGS. 2-5 show top views of embodiments of the present disclosure;

FIG. 6 shows an exploded angled top view of an embodiment of the present disclosure;

FIGS. 7-12 show a bottom view of an absorbent article embodiment of the present disclosure;

FIGS. 13-14 each show a flow chart according to a method of manufacture according to an embodiment of the present disclosure;

FIG. 15 shows a schematic view of a prior art apparatus according to an embodiment of the present disclosure;

FIG. 16 shows a schematic view of an apparatus according to an embodiment of the present disclosure;

FIG. 17a shows an angled view of an apparatus according to an embodiment of the present disclosure;

FIG. 17b shows a side view of the apparatus in FIG. 17a; and

FIG. 18 shows an angled view of an apparatus according to an embodiment of the present disclosure.

### DETAILED DESCRIPTION

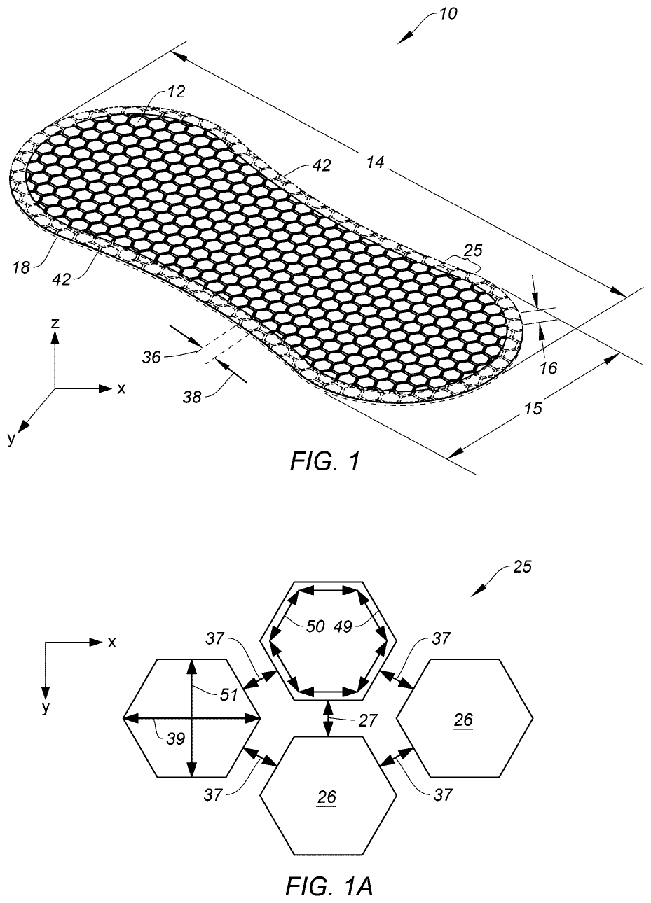

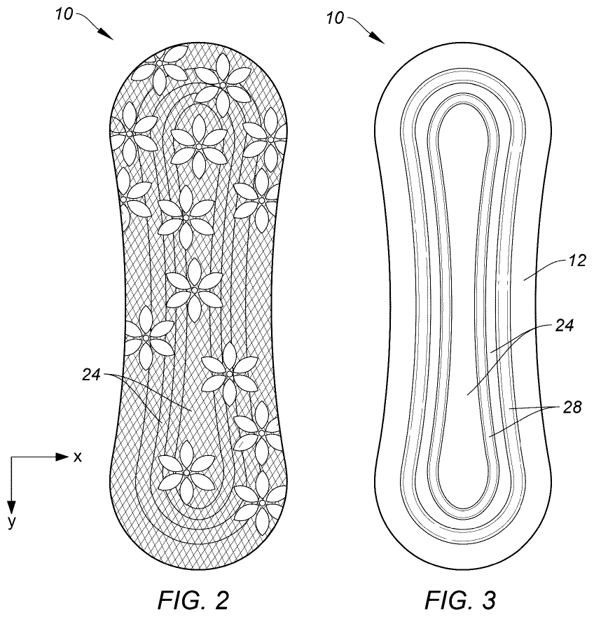

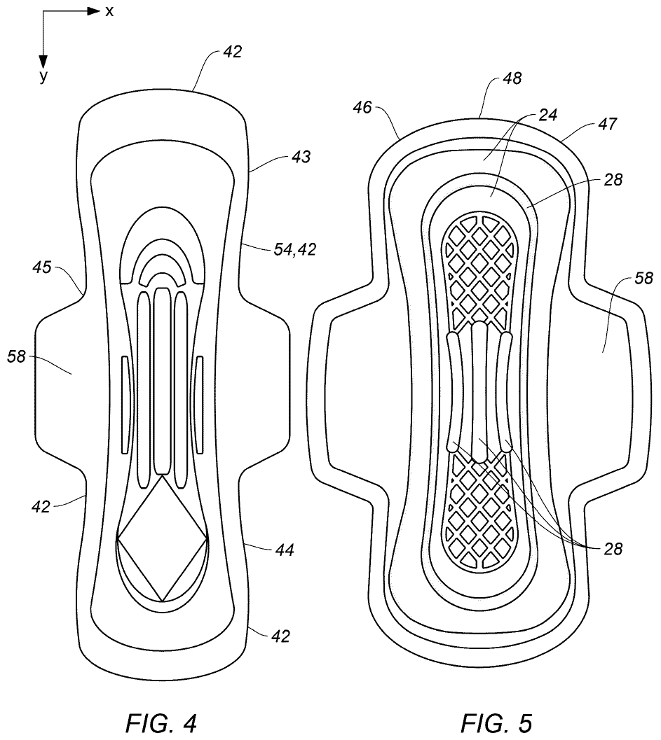

As exemplified in FIG. 1, an absorbent article (10) is provided. Coordinates are provided in the x, y, and z direction, and provide reference for appreciating the viewpoint of the subject described herein. The absorbent article has a length (14), a width (15), and a thickness (16), and generally includes a top-most (or body-facing) surface (11), often referred to as a cover layer (11). The absorbent article (10) has a backsheet (or back side, or barrier layer) (18) opposite the cover layer (11), and generally facing a garment worn by the user. The absorbent article has an absorbent core (20) structure between the cover layer (11) and the backsheet (18). Optionally, absorbent article (10) has a transfer layer (22) between the cover layer (11) and absorbent core (20).

The absorbent article (10) has many shapes, but as shown and preferably for at least ultra-thin products and liner products, a dog-bone, peanut-shape, hourglass, and/or barbell shape is provided. Alternative shapes include generally cylindrical, thong-shaped (such that the sides are not parallel and taper towards each other from the rear portion (or first end region 43) to the front portion (or second end region 44)), and/or hybrids thereof. The absorbent article (10) optionally has winged portions (one or more sets of winged portions emanating from the lateral sides of the central region (45) or the lateral side edges (42) of the second or rear region (44)) (58) that facilitate adherence to an undergarment or to itself for joining about the waist of a user. The absorbent article (10) optionally has a gathered waist region for pant-like or underwear-type absorbent articles (not shown).

Optionally, absorbent articles (10) included colored portions (28) and non-colored portions (24) to denote absorbent areas on the product, enhance the perception of depth of the absorbent article, and/or assist in masking fluids that ingratiate the product. Colored portions (28) are on the upper surface (12) of the cover layer (11), lower surface [that is opposite to the upper surface (12)] (13) of the cover layer (11), or on/within the absorbent core (20). Colored portions (28) can be varying colors but are preferably a darker color that is easier to see and optionally masks bodily discharge. Such colors are red, blue, violet, and/or green.

The cover layer (11) has a body facing surface (12) and a lower cover surface (13) opposite the body facing surface (12). The body facing surface (12), when used, is directed towards the body of a user such that the body facing surface (12) is at least partially in contact with the user, or at minimum, is the first portion of the absorbent article (10) that is ingratiate with bodily discharge such as fluid. Such fluid includes, for example, urine and/or menses.

In some embodiments, the cover layer (11) is a spunbound or spunlace material. The cover layer (11) includes nonwoven fibers and/or synthetic fibers. The cover layer (11) optionally has bicomponent fibers that are PE/PP. In one embodiment, the cover layer (11) includes a blend of fibers. In some embodiments, polypropylene, polyester, and/or polyethylene fibers are blended together. For instance, polyester and polypropylene fibers are blended, or polyethylene and polypropylene fibers are blended. Such ratios of fibers can be from about 1:3 to about 3:1, respectively, or said differently, between about 25% to about 75%, and between about 75% to about 25%, respectively. In some embodiments, the ratio of fibers can be approximately even, about 1:1, or about 50% each. In some embodiments, the cover layer (11) has a basis weight that is between about 20 gsm and about 50 gsm, or between about 25 gsm and about 30 gsm, or about 27 gsm. In some embodiments, the cover layer (11) is an imaged spunlace that is hydroentangled. In some embodiments, the cover layer (11) has a uniquely configured image or pattern. Cover materials are produced by manufacturers such as Suominen, Fitesa, and Shalag.

In some embodiments, the cover layer (11) is embossed. The embossing has one or more protrusions (26) separated by one or more channels (27). The one or more protrusions (26) have a protrusion height (38) and a protrusion width (or diameter) (39). The protrusion (27) honeycomb (25) structures are raised on the body facing surface (12) with respect to a cover layer base height (36). The protrusion height (38) is a first body facing height (38) and those terms are used interchangeably throughout. The base height (36) is a second body facing height (36) and those terms are used interchangeably throughout. In some embodiments, the embossing forms a pattern. In some embodiments, the cover layer (11) has an embossed pattern, the one or more protrusions (26) and the one or more channels (27) in such embossed pattern being a repeating pattern across at least a portion of the absorbent article, such as a first end region (43), a second end region (44), and/or a central region (45), and/or a first lateral region (46), a central length region (48), and/or a second lateral region (47) that is opposite the first lateral region (46). In some embodiments, the embossed pattern is a repeating pattern across substantially the entirety of the absorbent article (10).

In some embodiments, the cover layer (11) has a protrusions (26) and channels (27) forming a honeycomb (25) embossed pattern, the honeycomb shaped protrusions (26) (separated by channels (27)) being a repeating pattern across at least a portion of the absorbent article, such as a first end region (43), a second end region (44), and/or a central region (45), and/or a first lateral region (46), a central length region (48), and/or a second lateral region (27) that is opposite the first lateral region (46). In some embodiments, the honeycomb (25) pattern is a repeating pattern across substantially the entirety of the absorbent article (10). The individual hexagonal structures that are protrusions (26) in the cover layer (11) are separated by channels (27) between about 0.0125 inches and about 0.0375 inches, or between, about 0.025 inches. The individual hexagonal structures in the cover layer (11) have a protrusion height (38) (or the difference between first body facing height (38) and the second body facing height (36)) of between about 0.020 inches and about 0.040 inches, and more preferably, about 0.030 inches. The distance between the first body facing height (38) and the second body facing height (36) is at least 0.010 inches and is up to about 1 mm. Each side (49) of the protrusion (26), or in the exemplary embodiment, the side (49) of the individual hexagonal protrusion (26) has a protrusion length (50) of between about 0.080 inches and about 0.10 inches, or about 0.090 inches. The overall protrusion length (51) of the individual hexagonal protrusion (26) as defined by the midpoint of an exterior side to the midpoint of an opposite exterior side, thereby forming a perpendicular bisector between both sides, is between about 0.15 inches and about 0.20 inches, and more preferably about 0.16 inches to about 0.18 inches, or about 0.17 inches. The width/diameter (39) of the individual hexagonal protrusion (26) is between about 0.20 inches and about 0.30 inches, or between about 0.21 inches to about 0.28 inches.

The absorbent core (20) includes one or more layers referred to as strata (30). The absorbent core (20) is optionally an airlaid structure, wherein material is deposited onto a substrate. The absorbent core (20) has an upper core surface (21) that is adjacent the lower cover surface (13), and a lower core surface (23) that is adjacent the backsheet upper surface (17). Absorbent core structures can be purchased from manufacturers such as EAM and Glatfelter.

The absorbent core (20) includes one or more of the following: non-woven materials, fiber, pulp, absorbent material, and optionally adhesive material. The fiber or pulp material is between about 40% and about 95% of the total weight of the absorbent core (20). In some embodiments, the fiber material is between about 60% and 90% of the total weight of the absorbent core (20). In some embodiments, the fiber material is between about 80% and 90% of the total weight of the absorbent core (20).

In absorbent core (20) and/or one or more strata (30) therein having both pulp and bicomponent fibers, the ratio of pulp to bicomponent fibers is between about 3.5:1 and about 30:1. In a first embodiment, the ratio of pulp to bicomponent fibers is between about 3.5:1 and about 8:1. In a second embodiment, the ratio of pulp to bicomponent fibers between about 10:1 and about 40:1. In such embodiments, the pulp material is between about 40% and about 75% of the total weight of the absorbent core (20). In some embodiments, the pulp material is between about 40% and about 60% of the total weight of the absorbent core (20). In some embodiments, the pulp material is between about 55% and about 75% of the total weight of the absorbent core (20). In such embodiments, the bicomponent fiber is between 1% and 15% of the total weight of the absorbent core (20). In some embodiments, the bicomponent fiber is between 1% and 10% of the total weight of the absorbent core (20). In some embodiments, the bicomponent fiber is between 5% and 15% of the total weight total weight total weight total weight total weight total weight of the absorbent core (20).

In some embodiments, the absorbent core (20) includes glue that is between about 0.5% and about 10% of the total weight of the absorbent core (20).

In some embodiments, the ratio of pulp to bicomponent fibers between about 10:1 and about 40:1, where the pulp material is between about 55% and about 75% of the total weight of the absorbent core (20), and the bicomponent fiber is between 5% and 15% of the total weight of the absorbent core (20).

Pulp in the absorbent core (20) is treated or untreated, or a mixture of both treated and untreated. Such pulp is sold by, for example, manufacturers GP Cellulose or International Paper.

Bicomponent fibers are PE-PET and are sold by, for example, GP Cellulose or International Paper.

Glue used as a binder material for the absorbent core (20). One type of binder material is a dispersion binder. The dispersion binder is latex, vinyl acetate and/or ethylene. Some dispersion binders of the present disclosure are aqueous polymer dispersions.

Super absorbent material used in the absorbent core (20) is sold by Sumitomo, Nippon Shokubai, BASF, and Evonik. For example, some super absorbent polymer grades sold by Sumitomo include SA55, SA60, SA60N type II, SA65, SA65s, SA70. Super absorbent material is between about 5% and about 40% of the total weight of the absorbent core (20).

The backsheet (18) has a backsheet upper surface (17) that is opposite the backsheet garment facing surface (19). The backsheet upper surface (17) is adjacent the absorbent core (20) and more specifically, the lower core surface (23) of the absorbent core (20). The backsheet garment facing surface (19) is typically positioned adjacent the undergarment worn by a user. The backsheet garment facing surface (19) has releasable adhesive enabling temporary attachment to the undergarment, such that the backsheet garment facing surface (19) can be separated from the undergarment when absorbent article (10) needs to be replaced.

The positioning adhesive is applied in at least one stripe (32) to the backsheet garment facing surface (19) [that can be the back layer (34) of the absorbent article (10)], which is optionally the backsheet. More specifically, the garment facing surface (19) of the backsheet (18) has adhesive applied in a pattern such as at least one stripe (or line) (32). Optionally, the positioning adhesive includes multiple stripes (32) that are covered by one or more pieces of release material (52) such as release paper, overwrap, or a wrapper until the absorbent article (10) is ready to be applied to a garment for use. In some embodiments, between 1 and between about 50 lines (or stripes) (32) of adhesive are applied, such that the stripes (32) run the parallel to the length (14) of the absorbent article (10) or are perpendicular to the length (14). In some embodiments, between 2 and between about 5 lines (32) of adhesive are applied, such that the stripes (32) run the parallel to the length (14) of the absorbent article (10) or are perpendicular to the length (14). In some embodiments, 2 lines (32) of adhesive are applied, such that the lines (32) run the parallel to the length (14) of the absorbent article (10). In alternate embodiments, positioning adhesive is applied in a rectangular or square pattern, at least about the ends of the absorbent article. In certain embodiments, between 20 and 28 stripes (32) are applied perpendicularly to the length (14) of the absorbent article (10). In certain embodiments, between 30 and 38 stripes (32) are applied perpendicularly to the length (14) of the absorbent article (10). In certain embodiments, between 40 and 48 stripes (32) are applied perpendicularly to the length (14) of the absorbent article (10). In certain embodiments where more than one nozzle is being used, applying an even number of stripes (32) is preferred such that each nozzle is configured to spray the same pattern or same amount of adhesive. In certain embodiments, between 2 and 20 vertical stripes (32) that run parallel to the length (14) absorbent article (10) is preferred. In certain embodiments, the length of the stripes (32) is uniform such that all the stripes (32) have the same length. In certain embodiments, the stripes (32) have the same width such that all the stripes (32) are uniform. In certain embodiments, the spacing (40) between stripes (32) is between 1 mm and about 12 mm, or between 2 mm and about 10 mm, or between 2 mm and about 4 mm. In certain embodiments, the stripes (32) extend to within ¼″ and about 2.5″ of the edge of the absorbent article (10). In certain embodiments, the amount of adhesive applied to the back layer (34) of the absorbent article (10) is between about 20% and about 60% of the total surface area of the back layer (34), or between about 25% and about 45%, or between about 25% and about 35%. In certain embodiments, the length of the stripes (32) varies such that a first nozzle applies stripes (32) of a first length, and a second nozzle applies stripes (32) of a second length that is different from the first length

The absorbent article (10) with adhesive on the adhesive stripes (32) has an adhesive strength between 100 gf and 300 gf, or at least about 120 gf and up to about 300 gf, or at least about 150 gf and up to about 300 gf, or between about 120 gf and about 200 gf.

The mass of adhesive applied is between about 0.1 g to about 0.5 g, or between about 0.1 g and about 0.3 g, or between about 0.12 g and about 0.25 g, or between about 0.12 g and about 0.21 g.

In some embodiments, one or more wings (58) extend outwardly from the lateral edges (42) and are attachable to the undergarment. The wings (58) have a releasable adhesive enabling the wings (58) to temporarily adhere to the undergarment such that the wings (58) can be separated from the undergarment when absorbent article (10) needs to be replaced. In embodiments having releasable adhesive or pressure sensitive adhesive on the backsheet garment facing surface (19) and/or wings (58), the releasable adhesive is covered by release material (52) such as release paper and/or an overwrap or wrapper (52) to keep the absorbent article (10) from sticking to other items prior to use.

The absorbent article (10) has a length (14), width (15) and thickness (16), where the length (14) is between about 150 mm to about 330 mm, and the width varies between about 45 mm to about 150 mm (at its widest portion). The total thickness (15) of the cover (11), the optional transfer layer (22), and the absorbent core (20), is between about 0.9 mm and about 5 mm, or between about 1.5 mm and about 3.3 mm, and more preferably between 2.5 mm and about 3.3 mm. The cover (11), the optional transfer layer (22), and absorbent core (20), has a total weight of between about 1.3 to about 23 g, or between about 1.3 g and 6 g, or between about 1.3 g and about 2.8 g, or between about 7 g and about 23 g, or between about 7 g and about 10 g. The cover (11), the optional transfer layer (22), and absorbent core (22), has a total basis weight (gsm) of less than about 240 gsm, or between about 170 gsm and about 220 gsm, or between about 180 gsm and about 200 gsm.

The absorbent core (20) has a total weight of between about 2 g and about 5 g, or between about 3 g and about 4 g, or about 3.3 g to about 3.5 g. Having a more robust core material is preferred where the cover material is reduced in weight, where such combination of the absorbent core (20) and cover layer (11) enable a reduced thickness absorbent article (10). For example, the total thickness (15) of the absorbent article (10) is less than about 3 mm. Such thickness (15), in combination with structure and materials, enables and imparts advantageous properties and features contemplated throughout the present disclosure.

Table 1 below shows the improved properties achieved with the teachings of the present disclosure, using procedures generally in-line with the above-noted procedures:

TABLE 1
Comparison of Mass of Current Exemplary Product and New Embodiment

| | | Mass of | Mass of |
|---|---|---|---|
| Function | | STAYFREE | New Ultra Thin |
| (cover layer, | | Ultra Thin | Embodiment |
| transfer layer, | | with Wings | with |
| core materials | | and Odor | Wings and Odor |
| (Pulp and SAP) | | Neutralizer, | Neutralizer, |
| barrier film) | Description | Regular (g) | Regular (g) |
| Cover Layer (11) | Spunbond 18 gsm | 0.46 | 0.46 |
| | PE/PP Bico Oval | | |
| | Bond Pattern | | |
| Pulp | Untreated Fluff | 3.80 | 3.80 |
| SAP | Super Absorbent | 0.50 | 0.50 |
| | Polymer | | |
| Barrier Layer (18) | Polypropylene | 0.50 | 0.50 |
| (backsheet (19) or | 17 GSM White | | |
| back layer (34)) | | | |
| Pouch Overwrap | 19.5 gsm Printed | 0.59 | 0.59 |
| Film (52) | Polyethylene Film | | |
| Odor Neutralizer | Proprietary | 0.008 | 0.008 |
| Laminating | H20080 | 0.253 | 0.253 |
| Adhesive | | | |
| Positioning | H20028 | 0.141 | 0.141 |
| Adhesive | | | |
| Transfer | Airlaid 90 GSM | 0.91 | 0.91 |
| Layer (22) | | | |
| Blue Ink | Proprietary | 0.0009 | 0.0009 |
| Make-up Solution | Proprietary | 0.0000 | 0.0000 |
| Central Release | 37 GSM | 0.39 | 0.00 |
| Paper | | | |
| Wing | 37 GSM | 0.12 | 0.12 |
| Release Paper | | | |

| | | |
|---|---|---|
| Total Mass (g) | 7.67 | 7.28 |

As shown, by eliminating the need for the central release paper (and thus having only the absorbent article (10) and its wrapper (52), the total mass (shown above in the bottom row of Table 1) is reduced by up to about 10%, or between about 5% and 10%, or between about 8% and 10%. The reduction in material and mass leads to cost improvements as well as having a positive impact on the environment, as raw materials do not have to be harvested and/or made and/or processed, the raw material does not have to be packaged and transported, less energy is required to fully assemble the absorbent article (10), and less mass for shipping to distribution centers, customers, and/or consumers.

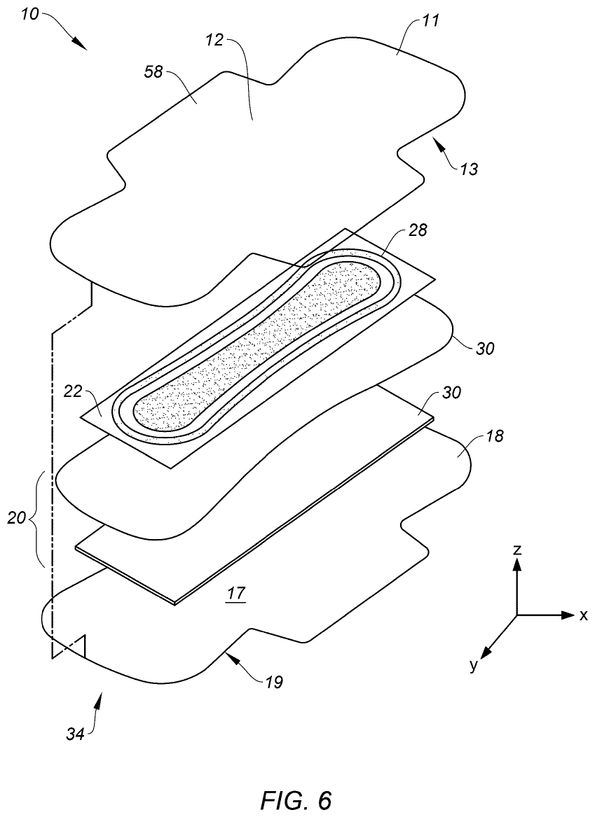

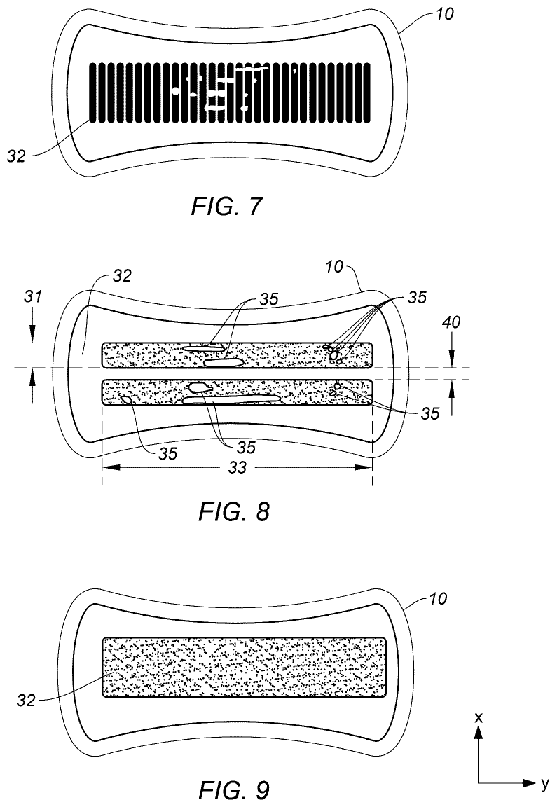

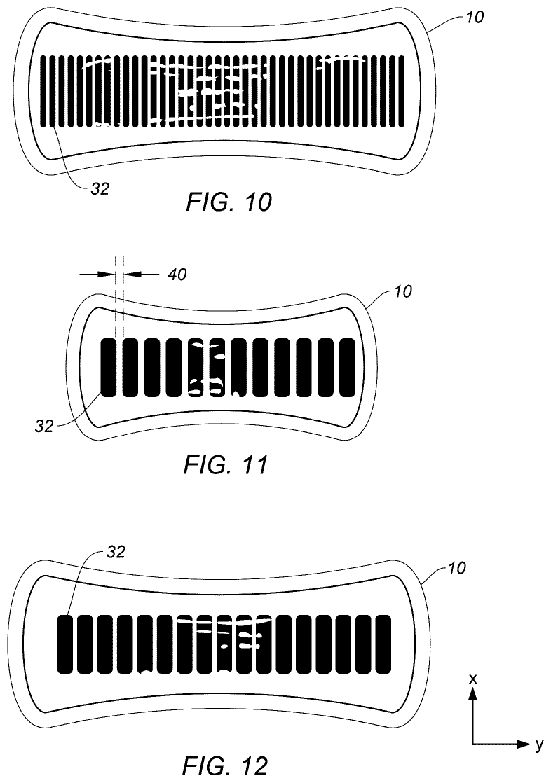

In another aspect of the present disclosure, a process and apparatus of the present disclosure provide improved consistency of adhesive within the adhesive stripe(s) (32) for embossed absorbent articles (10). Prior processes and apparatus' were unable to consistently apply adhesive in a configuration of one or more stripes (32), to which the present disclosure is able to achieve. As such, the absorbent article (10) of the present disclosure includes one or more adhesive stripes (32) that has a substantially uniform amount of adhesive applied throughout an individual adhesive stripe (32), in at least one of the length (33) and thickness (31) of the adhesive stripe (32). As such, the absorbent article (10) of the present disclosure includes one or more adhesive stripes (32) that has a substantially uniform amount of adhesive applied throughout all of the one more adhesive stripes (32) on the absorbent article (10), in at least one of the length (33) and thickness (31) of such one or more adhesive stripes (32). In some embodiments, the amount of surface area of an adhesive stripe (32) (as viewed by looking at the adhesive stripe (32) as it is positioned on the back layer (34) of the absorbent article (10), such as in FIGS. 6-8) that is covered by adhesive (such that none of the back layer (34) is uncovered within the adhesive stripe (32)) is greater than about 75%, or is greater than about 80%, or is greater than about 90%, or is greater than about 95%, or is greater than about 97%, or is greater than about 98% of the total size of the adhesive stripe (32). FIG. 8 exemplifies the portions of the stripes (32) that are not covered by adhesive with reference numeral (35), whereas stripes (32) are otherwise covered with adhesive. In some embodiments, the amount of surface area of all of the one or more adhesive stripes (32) (as viewed by looking at the adhesive stripe (32) as it is positioned on the backside of the absorbent article (10), such as in FIGS. 6-8) that is covered by adhesive (such that none of the back layer (34) is uncovered within the adhesive stripe (32)) is greater than about 75%, or is greater than about 80%, or is greater than about 90%, or is greater than about 95%, or is greater than about 97%, or is greater than about 98% of the total size of the adhesive stripe (32). Such measurements can be done with computer software and either high resolution cameras or microscopes, wherein the total surface area of the adhesive stripe (32) is measured. The surface area of the adhesive stripe (32) covered by adhesive is also measured, or alternatively, the surface area of the adhesive stripe that is not covered (35) by adhesive is measured. The percentage of surface area of an adhesive stripe (32) covered by adhesive can then be calculated by dividing (a) the surface area of the adhesive stripe (32) that is covered by the adhesive by (b) the total surface area of the adhesive stripe (32).

The absorbent article (10) according to the present disclosure is made possible by a unique manufacturing apparatus including a lobed roller (82). Currently, ultra thin absorbent articles (10) having an embossed cover sheet or otherwise having ribs, channels, and/or protrusions creating a varied topography on the cover sheet (11), have positioning adhesive sprayed on either the backsheet (18) of the pad or the release paper. In either event, the absorbent article (10) and the release paper are thereafter assembled such that the release paper is adhered onto the backsheet of the ultra thin article by way of a nip (76) defined by two rollers. The applicants discovered that applying positioning adhesive in a desired shape or pattern on an ultra-thin absorbent article (10) (having the aforementioned embossing or other topographical variation) and achieving a consistent spread of adhesive along the entirety (width and length) of each portion of the shape or pattern of the adhesive after being applied to film material such as that in a wrapper or overwrap was not achievable with current rollers having a uniform circumference. The applicants surprisingly realized that having uniform pressure applied to a structure with variable topography (by way of the cover layer) led to inconsistent application of the positioning adhesive. Counterintuitively, the applicants created an asymmetric roller, or lobed roller (82), that would enable discontinuous pressure over the length (14) of the absorbent article (10), where a release of pressure during the assembly of the release material (52) enabling the material to be applied and corrected to mitigate against inconsistent application of the adhesive from the release material (52) to the absorbent article (10).

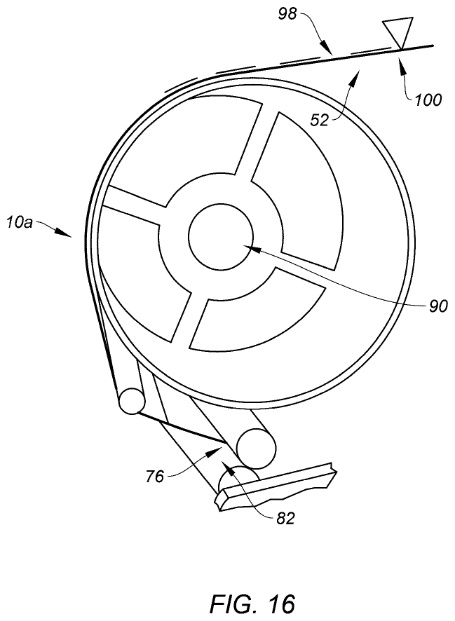

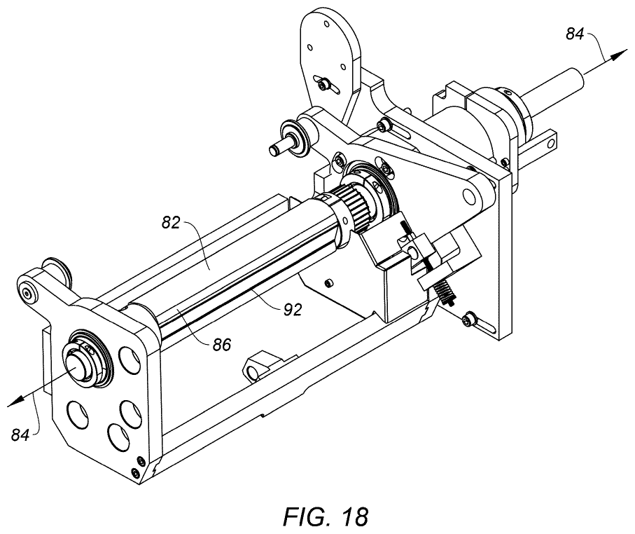

A method of applying adhesive to a release material (52) such as a wrapper, overwrap or release paper or onto a back layer (34) of an absorbent article (10) is provided. Each of these variants is described in series below, starting with applying the adhesive to the release material (52), then followed by applying the adhesive to the backside (34) of the absorbent article (10). FIGS. 15-18 show aspects of the method, products, and apparatus'.

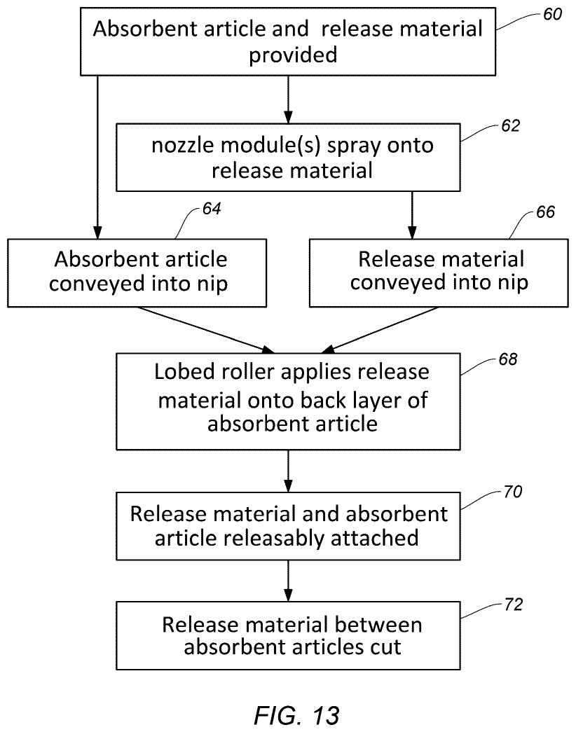

As exemplified in FIG. 13, the first step (60) of the method provides a release material (52) and an absorbent article (10). The absorbent article (10) is formed from at least a cover layer (11), a core (20), and a backsheet (19) together, and are assembled together either by pressure, heat, adhesive, or a combination thereof. The cover layer (11) may be formed to the backsheet (19), and/or each layer may be attached directly to its immediately adjacent layer. The absorbent article (10) has embossing such that it has one or more protrusions (26) separated by one or more channels (27). Alternatively, the absorbent article (10) has a varied topography on the cover layer (11) and thus also having protrusions (26), channels (27), ribs, recesses, etc.

The next step (62) in the method includes applying adhesive in a pattern to the release material (52) according to the present disclosure. The adhesive is applied by one or more nozzles. The nozzles are configured to spray one or more lines or stripes or patterns (32) of adhesive onto the release material (52) according to the present disclosure. After the adhesive applying step, the step (66) includes conveying the release material (52) i by at least a transfer drum (90) into a nip (76) having a gap size of between about 0.5 mm and about 4. mm, or between about 0.5 mm and about 3 mm, or about 1 mm. The release material (52) is conveyed such that the width of the material corresponds to the length (14) of the at least one absorbent article (10). The gap size can be adjusted to suit the size (i.e., the thickness) of the absorbent article (10). The gap size can be adjusted to accommodate different sized rollers suitable for different sized absorbent articles (10).

Simultaneously, step (64) occurs, where the absorbent article (10) is conveyed into the nip (76), where the absorbent article (10) is flat (or is not yet folded). In some embodiments, the length (14) of the absorbent article (10) is aligned with the width of the belt (78) or rotating drum (80). In certain embodiments, the absorbent article (10) is transferred by a belt (78) or by vacuum. In one embodiment, a rotating transfer drum (90) applies a vacuum pressure to the absorbent article (10).

As the absorbent article (10) is applied to the adhesive on the release material (52) within the nip (76) (step 68), pressure is applied by a lobed roller (82) thereby providing a secured but releasable connection between the release material (52) and the backside (34) of the absorbent article (10), culminating in step (70). Thereafter, the release material (52) now attached to the absorbent article (10) are passed through a cutting (or slitting) machine whereby each individual absorbent article (10) [with the release material (52)] is cut and separated for packing (step 72).

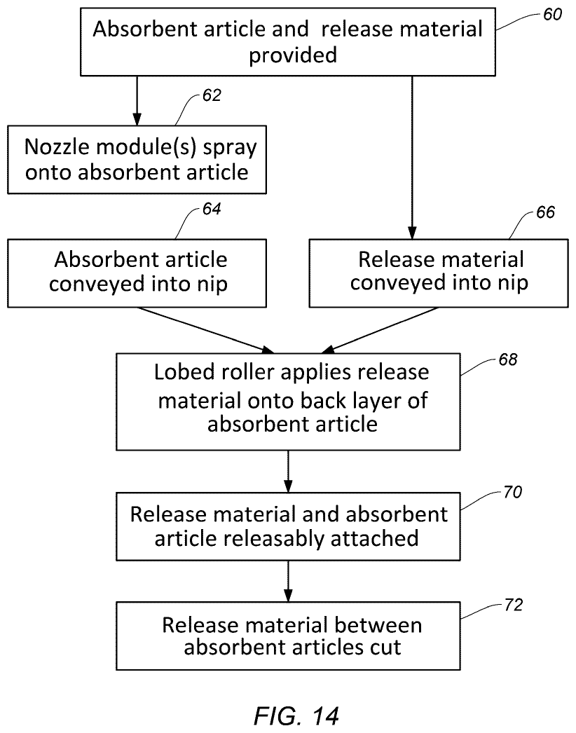

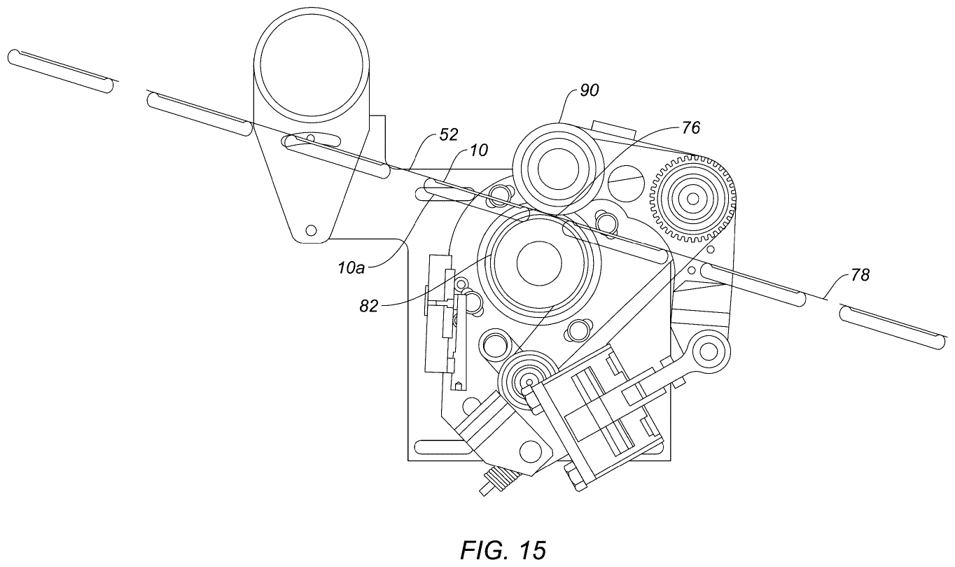

As exemplified in FIG. 14, the first step (60) of the method provides a release material (52) and an absorbent article (10). The absorbent article (10) is formed from at least a cover layer (11), a core (20), and a backsheet (19) together, and are assembled together either by pressure, heat, adhesive, or a combination thereof. The cover layer (11) may be formed to the backsheet (19), and/or each layer may be attached directly to its immediately adjacent layer. The absorbent article (10) has embossing such that it has one or more protrusions (26) separated by one or more channels (27). Alternatively, the absorbent article (10) has a varied topography on the cover layer (11) and thus also having protrusions (26), channels (27), ribs, recesses, etc.

The next step (62) in the method includes applying adhesive in a pattern to the absorbent article (10) according to the present disclosure. The adhesive is applied by one or more nozzles. The nozzles are configured to spray one or more lines or stripes or patterns (32) of adhesive onto the backside (34) of the absorbent article (10) according to the present disclosure. After the adhesive applying step, the step (66) includes conveying the absorbent article (10) by at least a transfer drum (90) into a nip (76) having a gap size of between about 0.5 mm and about 4. mm, or between about 0.5 mm and about 3 mm, or about 1 mm. The absorbent article (10) is conveyed such that the length (14) of the at least one absorbent article (10) corresponds to the width of the release material (52). The gap size can be adjusted to suit the size (i.e., the thickness) of the absorbent article (10). The gap size can be adjusted to accommodate different sized rollers suitable for different sized absorbent articles (10).

Simultaneously, step (64) occurs, where the release material (52)) is conveyed into the nip (76), where the absorbent article (10) is flat (or is not yet folded) (or the release material (52)). In some embodiments, the width of the release material (52) is aligned with the width of the belt (78) or rotating drum (80). In certain embodiments, the release material (52) is transferred by a belt (78) or by vacuum. In one embodiment, a rotating transfer drum (90) applies a vacuum pressure to the release material (52).

As the absorbent article (10) is applied to the adhesive on the backside (34) of the absorbent article (10) within the nip (76) (step 68), pressure is applied by a lobed roller (82) thereby providing a secured but releasable connection between the backside (34) of the absorbent article (10) and the release material (52), culminating in step (70). Thereafter, the release material (52) now attached to the absorbent article (10) are passed through a cutting (or slitting) machine whereby each individual absorbent article (10) [with the release material (52)] is cut and separated for packing (step 72) leading to a wrapped absorbent article (10a).

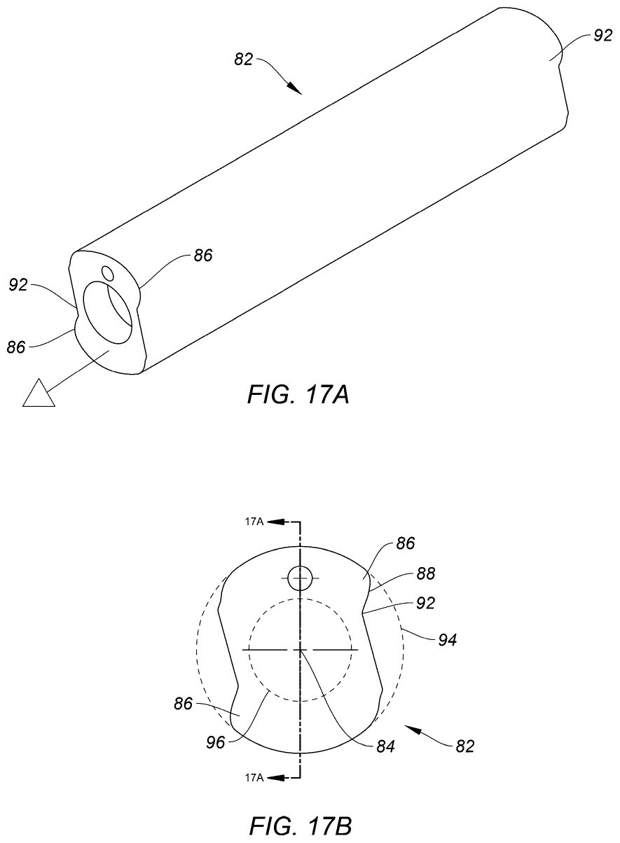

The absorbent article (10) and the release material (52) engage and pass through the nip (76) created by the lobed roller (82) and the transfer roller (90), where the transfer roller (90) is generally opposite the lobed roller (82) such that each of the two rollers (82, 90) is on the opposite side of the absorbent product (10) and the release material (52). The absorbent product (10) and the release material (52) engage the lobed roller (82) at the first portion (88) of the lobed portion (86). The lobed portion (86) of the lobed roller (82) is sized to be generally the width (15) of the absorbent article (10). The first portion (88) of the lobed roller (82) has a lead-in to mitigate against the absorbent article (10) from bunching and causing it to not properly sit on or mate with the adhesive applied to the release material (52). The lobed roller (82) has a pitch of between about 120 mm to about 150 mm, or about 130 mm to about 140 mm, or about 131 mm to about 136 mm. The lobed roller (82) as exemplified in FIG. 17b follows cam path (74) that is offset from the center axis (84) of the lobed roller (82).

The lobed roller (82) rotates at a speed substantially equal to the conveyed speed of the release material (52). In embodiments where the at least one absorbent article (10) is conveyed on a belt (78), the lobed roller (86) rotates at a speed substantially equal to the conveyed speed [of the belt (78)] of the at least one absorbent article (10). In embodiments where the at least one absorbent article (10) is conveyed by a transfer drum (90) utilizing a vacuum, the rotational speed of the lobed roller (86) equals the rotational speed of the transfer drum (90).

The lobed roller (86) is structured to have a first circumference (94) and a second circumference (96), where the first circumference (94) has a maximum diameter of the lobed portion (86) of the roller, where the second circumference (96) has a minimum diameter of the trough. The first circumference (94) forms a circumscribed circle about the entire lobed roller (82) including at least one lobed portion (86) and at least one trough (92). The second circumference (96) forms an inscribed circle about the entire lobed roller (82). In some embodiments, there is at least two lobed portions (86) and at least two troughs (92), where [forming basis for the second circumference (96)] troughs (92) separate each of the lobed portions (86).

An apparatus for applying a release material (52) to an absorbent article (10) is provided. The apparatus includes a drum having a rolling surface. The rolling surface is non-circular where a portion of rolling surface has a maximum diameter at a first circumference (94) that defines a circumscribed circle. The rolling surface is optionally lobed (86) such that a portion of the rolling surface has a second circumference (96) with a minimum diameter less than the first circumference (94) with a maximum diameter of the rolling surface defined by the lobed portion (86) of the rolling surface. The rolling surface is optionally cammed such that a portion of the rolling surface has a second circumference (96) with a minimum diameter less than the first circumference (94) with a maximum diameter of the rolling surface defined by the cammed portion of the rolling surface.

In this document, the terms “a” or “an” are used, as is common in patent documents, to include one or more than one. In this document, the term “or” is used to refer to a nonexclusive or, unless otherwise indicated. It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the present disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc., as they may be included, are used merely as labels, and are not intended to impose numerical requirements on their objects. In the Detailed Description provided above, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

1. An absorbent article having a length, a width, and a thickness, the length and width being in the x-y plane, comprising:
a cover layer having a body facing surface and a lower cover surface opposite to the body facing surface;
an absorbent core adjacent to the lower cover surface; and
a backsheet layer having a backsheet upper surface and a garment-facing surface opposite the backsheet upper surface, the garment-facing surface having at least some portions covered by a releasable adhesive;
wherein at least one of the cover layer and the absorbent core are embossed thereby creating a first body facing height that is greater than a second body facing height;
wherein the garment-facing surface has a releasable adhesive is applied in a plurality of stripes; and
wherein the plurality of stripes is between two stripes and fifty stripes.

2. The absorbent article according to claim 1, further comprising a release material comprising a semi-siliconized or a fully siliconized film material.

3. The absorbent article according to claim 1, wherein absorbent article is embossed to have at least one channel and up to about 25 channels.

4. The absorbent article according to claim 3, cover layer is embossed such that there are embossed protrusions on the body facing surface with respect to a cover layer base height.

5. The absorbent article according to claim 4, wherein the embossed protrusions have a protrusion height of between about 0.020 inches (0.5 mm) and about 0.040 inches (1.0 mm), and a protrusion width of between about 0.020 inches (0.5 mm) and about 0.040 inches (1.0 mm).

6. The absorbent article according to claim 5, further comprising at least one channel separating the embossed protrusions, wherein the channels have a channel width of between about 0.0125 inches (0.31 mm) and about 0.0375 inches (0.95 mm).

7. An absorbent article having a length, a width, and a thickness, the length and width being in the x-y plane, comprising:
a cover layer having a body facing surface and a lower cover surface opposite to the body facing surface;
an absorbent core adjacent to the lower cover surface; and
a backsheet layer having a backsheet upper surface and a garment-facing surface opposite the backsheet upper surface, the garment-facing surface having at least some portions covered by a releasable adhesive;
a release material being generally rectangular and having an upper edge, a left-most edge, a right-most edge generally opposite the left-most edge, and a lower edge generally opposite the upper edge, the release material having an article facing side and an outer side opposite to the article facing side wherein the article facing side and the outer side are defined by the upper edge, lateral edges, and lower edge, the release material having a release material length that is equal to or greater than the length of the absorbent article, the release material having a release material width that is equal to or greater than the width of the absorbent article, the release material comprising a semi-siliconized or fully-siliconized material or coating;
wherein at least one of the cover layer and the absorbent core are embossed thereby creating a first body facing height that is greater than a second body facing height;
wherein the release material has a releasable adhesive is applied in a plurality of stripes,
wherein the plurality of stripes is at least two stripes and up to about 50 stripes,
wherein the plurality of stripes have an adhesive surface area of up between about 20% and up to about 50% of the total surface area of the article facing side of the release material;
wherein the plurality of stripes have an adhesive surface area of between about 20% and up to about 50% of the total surface area of the garment-facing surface.

8. A method for applying adhesive to at least one ultra-thin absorbent article, comprising the steps of:
assemble the at least one ultra-thin absorbent article from at least a cover layer, a core having one or more portions or layers, and backsheet, wherein the backsheet has a garment facing surface that provides a backside of the at least one ultra-thin absorbent article;
wherein the cover layer is applied to and is adjacent the core, and the core is applied to and adjacent the backsheet, wherein the cover layer, core and backsheet are assembled into the at least one ultra-thin absorbent article by at least one of pressure, heat, adhesive, or a combination thereof;
wherein the at least one ultra-thin absorbent article is assembled such that the cover layer has a varied topography with one or more protrusions and one or more channels;
a release material is provided and is conveyed past one or more nozzles, wherein the nozzles spray adhesive onto the release material in one or more lines or patterns as the release material is conveyed;
convey the release material by at least a transfer drum into a nip having a gap size of between about 0.5 mm and about 5 mm;
wherein the release material is conveyed such that the width of the release material corresponds to the length of the at least one ultra-thin absorbent article;
simultaneously, convey the at least one ultra-thin absorbent article is conveyed into a nip on a transfer belt, where the at least one ultra-thin absorbent article is generally and substantially flat or not in a folded condition;
wherein the length of the at least one ultra-thin absorbent article is aligned with the width the transfer belt or rotating drum; and
as the at least one ultra-thin absorbent article is applied to the adhesive on the release material within the nip, pressure is applied by a lobed roller thereby providing a secured but releasable connection between the release material and the backside of the at least one ultra-thin absorbent article;
wherein the at least one ultra-thin absorbent article and the release material engage and pass through the nip created by the lobed roller and the transfer drum;
wherein the transfer drum is generally opposite the lobed roller such that each of the transfer drum and lobed roller are on the opposite side of the at least one ultra-thin absorbent article and the release material;
wherein the at least one ultra-thin absorbent article and the release material engage the lobed roller at the first portion of the lobed portion;
wherein a lobed portion of the lobed roller is sized to be generally the width of the at least one ultra-thin absorbent article;
wherein the first portion of the lobed roller has a lead-in to mitigate against the at least one ultra-thin absorbent article from bunching and causing the at least one ultra-thin absorbent article to not properly sit on or mate with the adhesive applied to the release material; and
wherein the lobed roller rotates at a speed substantially equal to the conveyed speed of the release material.

9. (canceled)

10. (canceled)

11. (canceled)

12. (canceled)

13. (canceled)

14. (canceled)

15. (canceled)

16. (canceled)
